(19) United States
(12) Patent Application Publication
(10) Pub. No.: US 2002/0151562 A1
(43) Pub. Date: Oct. 17, 2002

(54) Compositions and methods for treating allergic fungal sinusitis

(76) Inventor: Morton J. Seligman, Wyckoff, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/061,025

(22) Filed: Jan. 30, 2002

Related U.S. Application Data
(60) Provisional application No. 60/266,575, filed on Feb. 5, 2001.

(51) Int. Cl. A61K 31/47; A61K 31/41; A61L 9/04

(52) U.S. Cl. 514/311; 514/382; 424/45

(57) ABSTRACT

A method and composition for administering intranasally a composition comprising montelukast and pranlukast, and a pharmaceutically acceptable carrier in order to treat rhinitis, and asthma in humans is disclosed to treat allergic fungal sinusitis in humans.

### CROSS-REFERENCE TO RELATED APPLICATIONS

This application claims priority from U.S. Provisional Application No. 60/266,575 filed on Feb. 5, 2001 and which is hereby incorporated by reference.

### FIELD OF INVENTION

The present invention relates to compositions and methods for treating allergic fungal sinusitis in humans.

### BACKGROUND

Sinusitis, allergic rhinitis, and asthma are chronic inflammatory disorders of the airways. There are various treatments for these conditions which include corticosteroids (a type of anti-inflammatory medication found to be the most potent inhaled medication) antihistamines, decongestants, cromolyn sodium, nedocromil, and mild to moderate anti-inflammatory medications; long-acting and short-acting bata2-agonists; methylxanthines; anticholinergics; systemic corticosteroids; and anti-leukotriene agents used orally for asthma. It has been found that in cases where people have chronic rhinosinusitis, 93% of these patients have allergic fungal sinusitis (“AFS”). (U. Ponikau, “The Diagnosis and Incidence of Allergic Fungal Sinusitis,” Clinical Procedures, 1999; 74:877,881). Individuals with AFS have been found to be resistant to treatment with the usual noted medications. Therefore the use of many of the treatments which have been successful in treating sinusitis and other chronic inflammatory disorders of the airways have not been effective in treating allergic fungal sinusitis.

As set forth above, patients suffering from chronic sinusitis who did not respond to conventional treatments for this malady as recent studies indicated were found to be suffering from a malady attributable to a fungi which was the potential major antigenic trigger for this chronic sinusitis. Unlike other types of aeroallergens, fungi can colonize the sinus mucus resulting in much greater local concentrations of allergen. This is most convincingly demonstrated in “allergic fungal sinusitis” in which fungal hyphae are identified in allergic mucin. See DeShazo R D Swain R E. 1995, “Diagnostic criteria for allergic fungal sinusitis.” J Allergy Clin Immunol 96: 24-35; and Cody D T, Neel H B, Ferreiro J A, Roberts G D. 1994. “Allergic fungal sinusitis: the Mayo Clinic Experience.” Laryngoscope 104: 1074-9. Diagnostics of allergic fungal sinusitis can be carried out by the fungal colonization of sinus secretions and fungal sensitization by skin testing and the presence of these diseases can be a fairly common finding in this patient population. Such diagnosis of allergic fungal sinusitis are set forth in Ponikau, “The Diagnosis and Incidence of Allergic Fungal Sinusitis,” Clinical Procedures, 1999; 74:877,881.

Current pharmacologic management of asthma, sinusitis, and allergic rhinitis consists of corticosteroids, decongestants, antihistamines, anticholinergics and antibiotics. These treatments may result in severe side effects. Adrenal suppression, osteoporosis, growth retardation, arrhythmia, cataracts, and aggravation of glaucoma have occurred. In addition, as set forth above , these treatments have not been effective in alleviating allergic fungal sinusitis. Therefore an effective treatment for allergic fungal sinusitis without severe side effects is desired.

### BRIEF SUMMARY OF THE INVENTION

This invention provides for compositions and a method for intranasal administration of such composition containing montelukast, pranlukast or salts thereof for the effective treatment of allergic fungal sinusitis. In addition, the method provided by this invention allows for a low-dosage, low side effects, treatment of allergic fungal sinusitis with montelukast or pranlukast or salts thereof delivered intranasally with a pharmaceutically acceptable liquid carrier. The method eliminates the need for a fluorocarbon corticosteroid metered dose inhaler. Finally, the low dosage of the specific montelukast and pranlukast compositions administered intranasally provides for an effective treatment of allergic fungal sinusitis without substantial side effects common in the current treatments available for sinusitis and other chronic inflammatory disorders of the airways

### DETAILED DESCRIPTION OF THE INVENTION

In accordance with this invention it has been found that when patients suffering from allergic fungal sinusitis as well as those patients suffering from chronic sinusitis and asthma patients who have been unresponsive to normal treatment are administered intranasally an effective amount of a montelukast, pranlukast, or a pharmaceutically acceptable salt thereof, there is a substantial improvement in these patients in that the symptoms of these diseases lessened and in some patients these symptoms were entirely eliminated without many of the substantial side effects common in the current treatments for sinusitis and other chronic inflammatory disorders of the airways. It has been discovered that these effects are produced though the intranasal use of a montelukast, pranlukast or a pharmaceutically acceptable salt thereof.

In accordance with this invention the particular antagonists which accomplished this effect are montelukast which has the formula:

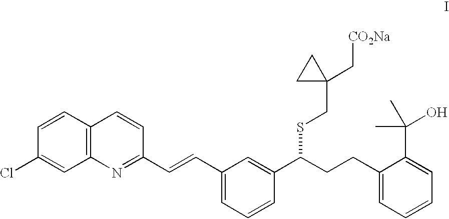

or its pharmaceutically acceptable salt, and pranlukast which has the formula:

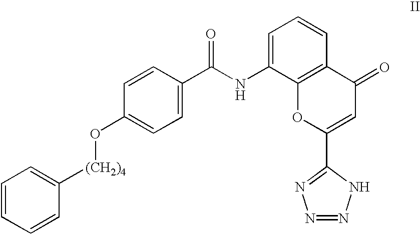

or its pharmaceutically acceptable salt.

By “pharmaceutically-acceptable salts” as used herein is meant salts of the compound of formula I and II which have the same general pharmacological properties as the compound from which they are derived, and which are acceptable from a toxicity viewpoint. Pharmaceutically acceptable salts of the compound of formula I include alkali metal (sodium and potassium), alkaline earth metal (calcium and magnesium), non-toxic heavy metal (stannous and indium), and ammonium and low molecular weight substituted ammonium (mono-, di- and triethanolamine) salts. In accordance with this invention, any pharmaceutically acceptable acid addition salt of the compound of formula II can be utilized. Illustrative inorganic acids which form suitable salts include hydrochloric, hydrobromic, sulphuric, and phosphoric acid and sulfate salts. Illustrative organic acids which form suitable salts include the mono-, di-, and tricarboxylic acids. Illustrative of such acids are, for example, acetic, glycolic, lactic, pyruvic, malonic, succinic, glutaric, fumaric, malic, tartaric, citric, ascorbic, maleic, hyroxymaleic, benzoic, hydrocybenzoic, pheynlacetic, cinnamic, salicylic, 2-phyenoxybenzoic, and sulfonic acids such as p-toluenesulfonic acid, methanesulfonic acid and 2-hydroxyethanesulfonic acid.

In accordance with this invention the compound of formula I or the compound of formula II or salts thereof are administered intranasally via a liquid composition embodying the compound of formula I, the compound of formula II or salts thereof and a liquid pharmaceutically acceptable carrier for intranasal administration. Any conventional liquid pharmaceutically-acceptable carrier for intranasal administration can be utilized, particularly saline.

The standard way for diagnosing allergic fungal sinusitis is set forth in the above-mentioned De Shazo, RD Swain publication in the J. Allergy Clin Immunol 96: pages 14 through 39 and the above-mentioned Ponikau, et al. publication in Clinic Proc. 74: 87-84. As seen from these publications, allergic fungal sinusitis is the probable cause for no response to treatment for chronic sinusitis and allergic rhinitis. The use of these specific compounds of formula I or II or their salts, is especially effective in treating the patients who have been non-responsive to conventional types of treatment for acute and chronic sinusitis and allergic rhinitis. This is true especially since the above publications demonstrate that the cause of this non-responsiveness is that these patients, while showing the symptoms of acute and chronic sinusitis and allergic rhinitis, are really suffering from allergic fungal sinusitis.

In the Ponikau study cited above, 93% of individuals who suffer from chronic rhinosinusitis have been found to also suffer from allergic fungal sinusitis. Since the 34 people treated in the study of this invention have been found to have chronic rhinosinusitis and have failed to respond to the conventional treatment, a similar percentage of the 34 individuals treated could also suffer from allergic fungal sinusitis. In accordance with this invention, improvement in these non-responders was noticed in their condition with the intranasal administration of the compound of formula I at a very low dosage.

In order to provide treatment in accordance with this invention, the composition containing the compound of formula I, II or their salts and a composition containing the compound of formula I, II or their salts are delivered to the patient by intranasal administration at an effective dose to relieve these symptoms of allergic fungal sinusitis. With respect to patients suffering from allergic fungal sinusitis these systems were not alleviated utilizing other standard treatments such as by means of corticosteroids. However when utilizing the intranasal composition of this invention, these symptoms were alleviated. The symptoms which were alleviated include relieving nasal congestion, sinus pain, postnasal drip, throat pain, serous otitis media, headaches, trouble breathing, and reduction in snoring volume were reduced. In cases of those asthma patients who have been resistant to other types of therapies, the use of the claimed compound of this invention substantially reduce and in many cases eliminate these symptoms.

In accordance with this invention, the dose which is effective for the treatment of this invention can be adjusted to individual needs. However, it is generally preferred to utilize a daily dose of from about 0.03 mg. to about 10.0 mg. which can be administered through one or repeated administrations of an intranasal spray. Generally it is preferred to utilize a daily dosage of from about 0.3 to about 1.8 mg. per day of the montelukast and the pranlukast compounds in accordance with this invention. While these dosage levels are based upon a 50 to 75 kg. adult they can be administered to children as well as to adults above this weight depending upon the severity of the system and the physician's judgment.

In accordance with this invention, the nasal sprays containing the compound of formula I can be administered without harmful effects at these low dosages. Generally some recovery can be seen within one day after application. In general, these sprays have been utilized on a daily basis for approximately two months and even longer with continued alleviation and in many cases complete removal of these symptoms. In accordance with this invention, any conventional intranasal spray composition can be prepared by mixing the compounds of formula I or II or their salts in a standard liquid carrier such as saline for use in intranasal sprays. Generally, these liquid compositions contain these compounds or their salts at a concentration of about 0.01 mg. to 10.0 mg. per milliliter of the composition. Preferably for about 0.03 mg. to 6.0 mg. per milliliter. The composition of this invention is a form suitable to provide a liquid spray composition which delivers an amount of the compounds of formula I or II or their salts for a given day or for a given series of days. The can be done with respect to a given day by mixing a given day's amount into the saline solution and allowing the patient to spray this given amount in a given day. On the other hand, the series of dosages for a series of days can be prepared by measuring the total dose amount of the said compounds into saline solution and administering this mixture in a metered bottle which can indicate the amount to be given in a single day. The sprays containing the liquid composition of this invention contain from 50 to 120 metered sprays. Generally, it is preferred to spray the composition 8 times a day to deliver nasally from about 0.03 mg. to 10 mg. of the active ingredient to the patient daily. Each of these sprays being administered twice, once in each nostril.

Therefore the preparation of this invention can be a liquid adopted for administration as spray. These liquid preparations such as those based on nasal formulations may include the conventional auxiliary agents generally found in nasal sprays. For example, Ph buffing system, preferably a buffer such as phosphate, citrate or acetate buffer from a preservative in the osmotic pressure controlling agents (e.g. surfactants or sodium chloride. The sprays can also include nonionic surfactants which enhance nasal absorption of the drug such as polysorbate-80 together with one or more pharmaceutically carriers and if desired other therapeutic ingredients. The carriers must be “acceptable” in the sense of being compatible with other ingredients for formulation not deleterious to the recipient thereof. Such carriers are well-known to those skilled in the art of pharmacology. These formulations may be prepared by any of the methods well-known in the art of pharmacy. The preparations of this invention may be used in any dosage dispensing device adopted for intranasal administration. These devices should be constructed with a view to ascertaining optimum metering accuracy compatibility of its constructive elements, such as container valve and actuator with the nasal formulation could be based on a mechanical system.

### EXAMPLES

### EXAMPLE 1

Fifty-four patients were selected on the basis of their failure to respond to intranasal corticosteroids for the treatment of acute and chronic sinusitis and allergic rhinitis and positive skin tests to fungi. Based upon this, these people were diagnosed to have allergic fungal sinusitis resistant to treatment. The age range was 6 years to 71 years.

The saline nasal spray suspension of the sodium salt of montelukast was prepared by grinding 10 mg. with mortar and pestle and then added the result and powder was added to 10 ml. of normal sterile saline in a typical polyethylene nasal spray container. Each patient was instructed to use two sprays in each nostril twice daily, AM and PM for a total of four sprays per nostril per day. Treatment lasted for two months. However many patients on there own continued to use the nasal spray.

After beginning the treatment of intranasal administration of the sodium salt of montelukast, the patients were instructed to record improvement in their symptoms and whether improvement lasted. The patients were asked a series of questions regarding their symptoms before and after the treatment with montelukast. Therefore, the status of the patients' symptoms were documented over a period of two months.

At the end of two months forty-two of the fifty-two patients had positive results and alleviation of major symptoms and continuation of treatment was requested by these patients. The preparation was effective not only in relieving nasal congestions, sinus pain, post nasal drip, throat pain and serous otitis media, but it also helped their asthma, especially at night often eliminating the need for metered dose inhalers.

### Nasal Spray Study

The following statements were posed to the patients after treatment for two months with regard to their symptoms before and after the course of treatment. The patient was then asked to respond to the statement with either yes or no with regard to the questions prior to treatment, and with regard to questions after treatment, the patient had the following four categories to choose from: Marked Improvement, Improvement, No Change, Worse. The questions asked and the results are as follows: “When I walk or do simple chores, I have trouble breathing or I cough . . . ”

| “When I walk or do simple chores, I have trouble breathing | |
|---|---|
| or I cough . . . ” | |
| Total Number of Patients Treated: | 52 |
| Total Number of Patients with Trouble Breathing before Treatment: | 34 |
| Total Number of Patients Reporting Improvement of Breathing | 24 |
| Post-Treatment: | |
| “When I walk or do simple chores, I have trouble breathing | |
| or I cough . . . ” | |
| Total number of Patients Treated: | 52 |
| Total number of Patients that Avoided Activity due to Post Nasal | 25 |
| Drip, Nasal Decongestion and Sinus Pain Prior Treatment: | |
| Total Number of Patients Post Treatment that Reported Improvement: | 33 |
| “I have been unable to sleep through the night without coughing and | |
| choking attacks, post nasal drip and sinus pain.” | |
| Total Number of Patients Treated: | 52 |
| Total Number of Patients Prior to Treatment Reporting Sleeping | 29 |
| Trouble due to Coughing and Choking Attacks, Post Nasal Drip and | |
| Sinus Pain Prior Treatment: | |
| Total Number of Patients Post Treatment Reporting Improvement: | 28 |
| “Dust, pollen and pets cause nasal congestion, sinus pain, ear pain | |
| and dizziness” | |
| Total Number of Patients Treated: | 52 |
| Total Number of Patients Prior to Treatment Reporting Dust, Pollen, | 45 |
| and Pets cause Nasal Congestion, Sinus Pain, Ear Pain and Dizziness: | |
| Total Number of Patients Post Treatment Reporting Improvement: | 37 |
| “My sinus pain, post nasal drip and nasal congestion are worse in | |
| cold weather.” | |
| Total Number of Patients Tested: | 52 |
| Total Number of Patients Prior to Treatment Reporting Sinus Pain, | 21 |
| Post Nasal Drip and Nasal Congestion more Severe during Cold | |
| Whether Prior to Treatment: | |
| Total Number of Patients Post Treatment Reporting Improvement: | 14 |
| “My sinuses and nasal congestion are worse when I'm around | |
| tobacco smoke, fumes or strong odors.” | |
| Total Number of Patients Tested: | 52 |
| Total Number of Patients Prior to Treatment Reporting that Sinus and | 42 |
| Nasal Congestion more Severe around Tobacco Smoke, Fumes or | |
| Strong Odors Prior to Treatment: | |
| Total Number of Patients Reporting Improvement: | 24 |
| “When I catch a respiratory infection, it goes into my sinuses, | |
| ear and nose.” | |
| Total Number of Patients Treated: | 52 |
| Total Number of Patients Reporting Prior to Treatment that | 44 |
| Respiratory Infection often goes into Sinuses, Ears and Nose: | |
| Total Number of Patients Reporting Improvement: | 27 |
| “My allergy medicine doesn't control my nasal congestion, sinusitis, | |
| post nasal drip and ear congestion.” | |
| Total Number of Patients Treated: | 52 |
| Total Number of Patients that Reported Prior to Treatment that their | 42 |
| Allergy Medicine does not Control Nasal Congestion, Sinusitis, | |
| post Nasal Drip and Ear Congestion: | |
| Total Number of Patients Post Treatment that Reported Improvement: | 42 |
| “I worry that my sinus pain, post nasal drip and nasal congestion | |
| affects my health and makes it difficult to sleep.” | |
| Total Patients Tested: | 52 |
| Total Number of Patients Prior to Treatment that Worry that Sinus | 31 |
| Pain, Post Nasal Drip and Nasal Congestion Affect their Health and | |
| Makes it Difficult to Sleep: | |
| Total Number of Patients Post Treatment that Reported Improvement: | 30 |

Based on the results of this study, the intranasal administration of the sodium salt of montelukast was effective in treating allergic fungal sinusitis, allergic rhinitis, and asthma in the patients tested at an extremely low dose with no significant side effects. It should be noted that all of these patients were resistant to the other standard treatments including corticosteroids. Generally patients responding to this treatment had a substantial reduction in one or more of their symptoms of asthma.

### EXAMPLE 2

The study of Example 1 is conducted in the same manner with pranlukast yielding similar results to that obtained with montelukast sodium.

Some modifications and alternative embodiments of the invention will be apparent to those skilled in the art in view of the foregoing description. Accordingly, this description is to be construed as illustrative only and is for the purpose of teaching those skilled in the art how to carry out the invention. Details of the process my be varied substantially without departing from the concept of the invention and the exclusive use of all the modifications which come within the scope of the appended claim is reserved.

What is claimed is:

1. A method of treating patients suffering from allergic fungal sinusitis comprising administering intranasally to said patients an effective amount of liquid composition containing a compound of the formula 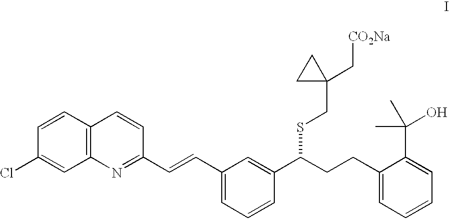
or a pharmaceutically acceptable salt,
and a pharmaceutically acceptable liquid carrier, said composition being intranasally administered to deliver an effective amount of said compound to treat said sinusitis.

2. The method of claim 2 wherein said compound is administered to a patient at a daily dosage of from about 0.03 mg. to 10 mg.

3. The method of claim 2 wherein said dose is from about 0.3 mg. to about 1.8 mg. per day.

4. A method of treating patients suffering from allergic fungal sinusitis comprising administering intranasally to said patents an effective amount of liquid composition containing a compound of the formula. 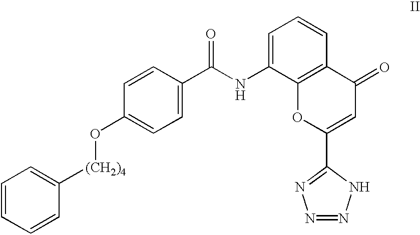
or a pharmaceutically acceptable salt,
and a pharmaceutically acceptable liquid carrier, said composition being administered to deliver an effective amount of said compound intranasally to treat said sinusitis.

5. The method of claim 4 wherein said compound is administered to a patient at a daily dosage of from about 0.03 mg. to 10 mg.

6. The method of claim 5 wherein said dose is from about 0.3 mg. to about 1.8 mg. per day.

7. A method for treating a patient suffering from a disease state which is sinusitis, allergic rhinitis or asthma who is non-responsive to conventional treatments for these disease states comprising administering intranasally to said patients an effective amount of liquid composition containing a compound of the formula 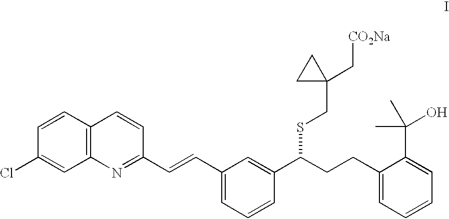
or a pharmaceutically acceptable salt,
and a pharmaceutically acceptable liquid carrier, said composition being administered to deliver an effective amount of said inhibitor intranasally to treat said sinusitis.

8. The method of claim 7 wherein said compound is administered to a patient at a daily dosage of from about 0.03 mg. to 10 mg.

9. The method of claim 8 wherein said dose is from about 0.3 mg. to about 1.8 mg. per day.

10. A method for treating a patient suffering from a disease state which is sinusitis, allergic rhinitis or asthma who is non-responsive to conventional treatments for these disease states comprising administering intranasally to said patients an effective amount of liquid composition containing a compound of the formula. 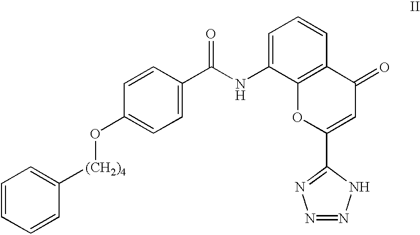
or a pharmaceutically acceptable salt,
and a pharmaceutically acceptable liquid carrier, said composition being administered to deliver an effective amount of said inhibitor intranasally to treat said sinusitis.

11. The method of claim 10 wherein said compound is administered to a patient at a daily dosage of from about 0.03 mg. to 10 mg.

12. The method of claim 11 wherein said dose is from about 0.3 mg. to about 1.8 mg. per day.

13. An intranasal spray comprising a liquid composition containing from 0.01 mg. to 10 mg. per ml. of a compound having the formula 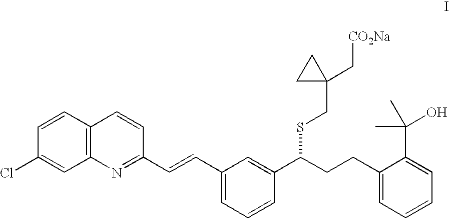
or a pharmaceutically acceptable salt thereof
and a liquid pharmaceutically acceptable carrier, said composition being in a form suitable to supply said compound intranasally to a patient as a spray in a daily amount of from 0.03 to 10 mg.

14. The spray of claim 13 wherein said carrier is saline.

15. The spray of claim 14 wherein said compound is present in an amount of from 0.3 to 6.0 mg. per ml. of the composition.

16. The spray of claim 13 wherein the composition is present in an amount to supply from 50 to 120 sprays to the patient.

17. An intranasal spray comprising a liquid composition containing from 0.01 mg. 10 mg. per ml. of a compound having the formula 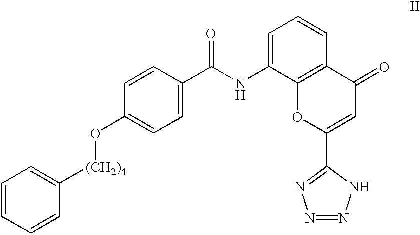
and a liquid pharmaceutically acceptable carrier, said composition being in a form suitable to supply through intranasal delivery said compound to a patient as a spray in a daily amount of from 0.03 to 10 mg.

18. The spray of claim 17 wherein said carrier is saline.

19. The spray of claim 18 wherein said compound is present in an amount of from 0.3 to 6 mg. per ml. of the composition.

20. The spray of claim 17 wherein the composition is present in an amount to supply from 50 to 120 sprays to the patient.
